(19) United States
(12) Patent Application Publication
(10) Pub. No.: US 2004/0122080 A1
(43) Pub. Date: Jun. 24, 2004

(54) Synergist combinations of retinoid receptor ligands and selected cytotoxic agents for treatment of cancer

(76) Inventors: Valerie S Vivat-Hannah, Andlau (FR); Matthew V Lorenzi, Philadelphia, PA (US); Marco M Gottardis, Princeton, NJ (US); Fred Christopher Zusi, Hamden, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/472,690

(22) PCT Filed: Mar. 22, 2002
(86) PCT No.: PCT/US02/08718

(51) Int. Cl. A61K 31/337; A61K 31/19

(52) U.S. Cl. 514/449; 514/568

(57) ABSTRACT

Chemotherapeutic combinations of selected cytotoxic agents and RARα/β selective agonists or RAR pan antagonists for use in treating cancer and lowering the effective cytotoxic dose of the selected cytotoxic agent are provided.

### RELATED APPLICATIONS

This application claims priority benefit under Title 35 § 119(e) of U.S. provisional Application No. 60/277,754, filed Mar. 22, 2001.

### FIELD OF THE INVENTION

The present invention relates to chemotherapeutic strategies for the treatment of cancer wherein retinoic acid receptor (RAR) ligands, in particular α/β selective agonists or RAR pan antagonists, are administered in combination with tubulin polymerizing agents such as taxanes. As demonstrated herein, RARα/β selective agonists and RAR pan antagonists are effective anti-cancer agents and exhibit reduced toxicity as compared to RAR pan agonists which also activate RARγ and natural retinoids such as all trans retinoic acid which activate RARγ and RXR. Further, the RARα/β selective agonists and RAR pan antagonists act synergistically with tubulin polymerizing agents, particularly taxanes, dramatically lowering the effective dose of taxane required to induce cytotoxicity of a wide range of tumor cell lines. It is believed that the synergy with RARα/β selective agonists is related in part to effects on Bcl-2 expression/phosphorylation as well as the activity of the JNK and AP1. Accordingly, similar synergies with RARα/β selective agonists are also expected with other agents which increase phosphorylation of Bcl-2.

### BACKGROUND OF THE INVENTION

The retinoids are biologically active derivatives of vitamin A that mediate a wide range of physiologic functions during both embryogenesis and adult life (Vitamin A in Health and Disease. Ed. Blomhoff, Institute for Nutrition Research, University of Oslo, Oslo Norway, 1994; Kastner et al. Cell 1995 83:859-869; Giguere et al. Ann. N.Y. Acad. Sci. 1996 785:12-22). The natural retinoids primarily consist of all trans retinoic acid (ATRA) and 9-cis retinoic acid, which are processed from vitamin A through its irreversible oxidation (Vitamin A in Health and Disease. Ed. Blomhoff, Institute for Nutrition Research, University of Oslo, Oslo Norway, 1994; Napli, J. L. Faseb J. 1996 10:993-1001). In addition, multiple synthetic ligands have been designed which retain many of the biological properties of the natural ligands.

At the cellular level, retinoid ligands can regulate the proliferation, differentiation and apoptosis of a wide variety of both normal and malignant cells (Vitamin A in Health and Disease. Ed. Blomhoff, Institute for Nutrition Research, University of Oslo, Oslo Norway, 1994; Lotan, R. Sem. in Cancer Biol. 1991 2:197-208; Love, J. M. and Gudas, L. J. Curr. Opinion in Cell Biol. 1994 6:825-831; Lotan, R. Faseb J. 1996 10:1031-1039; and Fitzgerald et al. Cancer Res. 1997 57:2642-2650). The biological activities of the retinoids are mediated by two classes of nuclear receptors, the retinoic acid receptors (RARs) and the retinoid X receptors (RXRs). Both the RAR and RXR classes are comprised of three isotypes designated α, β and γ, which are encoded by distinct genes (Chambon, P. Sem. in Cell Biol. 1994 5:115-125; Chambon, P. Faseb J. 1996 10:940-954). RAR and RXR are members of the nuclear hormone receptor (NHR) superfamily and act as ligand-dependent transcription factors, which can modulate target gene expression by binding as RAR/RXR heterodimeric complexes to the specific retinoid response element (RARE) DNA sequence, which consists of direct repeats of the consensus sequences PuG(G/A)(T/A)CA (n)₁₋₅ PuG(G/T)TCA (Gronemeyer, H., and Moras, D. Nature 1995 375:190-191). The activity of the RAR/RXR heterodimer can be repressive or stimulatory, depending upon its apo-(unliganded) or holo-(ligand-bound) conformation, respectively. The apo-conformation of RAR/RXR heterodimers is a permissive structure allowing protein interaction interfaces to initiate large co-repressor multi-protein complex formation including the recruitment of NCOR and histone deacetylases which together maintain the chromatin in a condensed transcriptionally inactive structure to repress target gene expression (Xu et al. Curr. Opinion in Genetic and Development 1999 9:140-147; Hu, X. and Lazar, M. A. Trends in Endocrin. and Metab. 2000 11:6-10; Robyr et al. Mol. Endocrin. 2000 14:329-347). In contrast, the binding of agonist ligand to RARs is associated with a conformational transition of the ligand binding domain (LBD; Renaud et al. Nature 1995 378:681-689) resulting in the destabilization of the corepressor complex and simultaneous recruitment of coactivators, including p160 protein family, CBP/p300 and the multiprotein complexes TRAP/DRIP/ARC. Some of these factors contain histone acetyltransferase activity allowing the decondensation of the chromatin required for the subsequent link of the holo-RAR/RXR heterodimer with the basal transcriptional complex to initiate target gene expression (Xu et al. Curr. Opinion in Genetic and Development 1999 9:140-147; Robyr et al. Mol. Endocrin. 2000 14:329-347).

ATRA and synthetic retinoid receptor ligands have been shown to promote tumor regression in a number of animal models of carcinogenesis and have shown efficacy in patients afflicted with acute promyelocytic leukemia (Chomienne et al. Faseb J 1996 10:1025-1030; Fenaux et al. Leukemia 2000 14:1371-1377). The combination of 13cis retinoic acid with paclitaxel or cisplatin and/or interferon-alpha has entered phase I/II trials for the treatment of head and neck squamous cell carcinomas, non small cell lung carcinomas and prostate cancer (Gravis et al. Anticancer Drugs 1999 10:369-374; DiPaola et al. J. Clin. Oncol. 1999 17:2213-2218). The RAR agonist, ALRT1550, potent inhibitor of AP1, has also been included in a phase I/II study on a heterogeneous population of 15 patients with advanced cancer, including non-small cell lung, thyroid, kidney, uterine leiomyosarcoma, prostate and adenoid cystic carcinoma (Soignet et al. Proc. Annu. Meet. Am. Soc. Clin. Oncol. 1998 17:A826).

Cytosine arabinoside in combination with paclitaxel and ATRA was also suggested as a combination chemotherapy for acute myeloblastic leukemia. More recently, ATRA has been shown to sensitize breast cancer cell lines to the cell killing effects of paclitaxel and Adriamycin (Wang et al. Cancer Res. 2000 60:2040-2048).

ATRA, 13cis retinoic acid, and ALRT1550 are all RAR pan agonists, meaning that they target and activate the α, β, and γ RAR isotypes. Further, ATRA is converted in situ into 9cis-RA which is a pan RAR and RXR agonist.

Of the three RAR isotypes, the RARβ-2 isotype has been associated with the tumor-suppressive effects of the retinoids. RARβ-2 arises from alternative promoter use of the RARβ gene promoter P2 (Chambon, P. Sem. In Cell Biol. 1994 5:115-125), which contains a strong RARE suggesting that RARβ expression is upregulated by retinoic acid and places RARβ2 as a potential target gene underlying the growth suppressive effects of retinoic acid. Consistent with a role in modulating cell growth, RARβ gene inactivation in F9 teratocarcinoma cells results in the loss of retinoic acid dependent growth arrest (Faria et al. J. Biol. Chem. 1999 274:26783-26788), whereas retroviral mediated expression of RARβ in breast cancer cells is associated with a strong enhancement of retinoic acid responsiveness in terms of growth inhibition and induction of apoptosis (Seewaldt et al. Cell Growth and Differentiation 1995 6:1077-1088). These observations highlight RARβ as an important mediator of retinoid action and suggest its modulation may be effective in reducing tumor cell growth.

However, loss of the RARβ2 expression during the early phases of cancer development including breast and lung tumors has been reported (Widschwendter et al. Cancer Res. 1997 57:4158-4161). This loss is believed to result from a repressive state of the chromatin induced by methylation and/or deacetylation to inactivate the RARβ2 promoter rather than genetic alteration (Arapshian et al. Oncogene 2000 19:4066-4070). A pure RARβ agonist had no effect on cell proliferation.

In contrast, the expression of RARα and -γ as well as RXRα are mostly unaltered in malignant tissues. However, the mucocutaneous toxic effects of retinoids which are considered as the major toxicity induced by retinoids and limits their use as therapeutic agents is associated with RARγ activity.

### SUMMARY OF THE INVENTION

An object of the present invention is to provide synergistic chemotherapeutic combinations for use in the treatment of cancer. In one embodiment of the present invention, the synergistic combination comprises a RARα/β selective agonist and a second agent which increases phosphorylation of Bcl-2, such as a tubulin polymerizing agent, preferably a taxane. In another embodiment of the present invention, the synergistic combination comprises a RAR pan antagonist and a tubulin polymerizing agent, preferably a taxane.

Another object of the present invention is to lower the effective dose of a selected cytotoxic agent required to induce cytotoxicity in tumor cells by administering the selected cytotoxic agent in combination with a RARα/β selective agonist or RAR pan antagonist.

Yet another object of the present invention is to provide methods of treating cancer in patients with these synergistic chemotherapeutic combinations. In one embodiment, a patient is administered a RARα/β selective agonist in combination with a second agent which increases phosphorylation of Bcl-2, such as a tubulin polymerizing agents, preferably a taxane. In another embodiment, the patient is administered a RAR pan antagonist in combination with a tubulin polymerizing agent, preferably a taxane.

### BRIEF DESCRIPTION OF THE FIGURES

FIG. 1 provides a synthetic scheme for exemplary RARα/β selective agonists useful in the present invention.

FIG. 2 provides a synthetic scheme for an exemplary RAR pan antagonist useful in the present invention.

### DETAILED DESCRIPTION OF THE INVENTION

The present invention relates to RARα/β selective agonists and RAR pan antagonists and their use in synergistically enhancing tumor cell sensitivity to selected cytotoxic agents.

For purposes of the present invention, by the term “RARα/β selective agonist” it is meant a compound which activates the α and β isotypes of the retinoic acid receptor but which exhibits little or no activation of, or actually inhibits, the γ isotype. Exemplary RARα/β selective agonists useful in the present invention are depicted in general in Formula I:

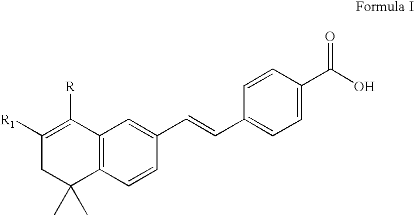

wherein R₁ is a hydrogen or methyl and R is cis-CH═CH—C(CH₃)₃ or cis-CH═CH—C(CH₃)₂CH₂CH₃. More specific exemplary RARα/β selective agonists are depicted in Formula Ia and Forula Ib:

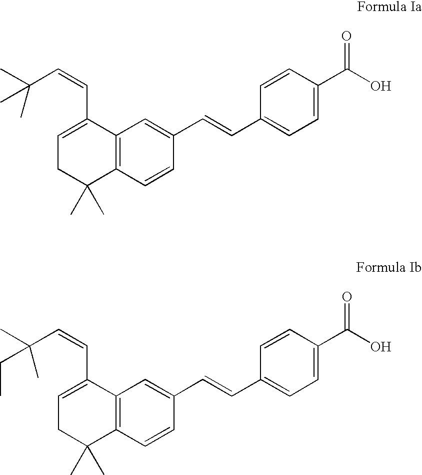

Formula Ia and the synthesis thereof is described in detail in WO 00/17147, the teachings of which are incorporated herein in their entirety, for use in treatment of skin disorders and as an antitumor agent. As will be understood by those of skill in the art upon reading this disclosure, other RARα/β selective agonists than those specifically exemplified herein can also be used in the present invention. For example, additional exemplary compounds exhibiting selectivity to RARα and RARβ are described in WO 97/48672. The ability of a compound to activate the α and β isotypes of the retinoic acid receptor while exhibiting little or no activation of the γ isotype can be determined routinely in accordance with procedures such as described in WO 97/48672 and Gehin et al. (Chemistry and Biology 1999 6:519-529).

By the term “RAR pan antagonist” it is meant a compound which inhibits the α, β, and γ isotypes of the retinoic acid receptor. An exemplary RAR pan antagonist is depicted in Formula II:

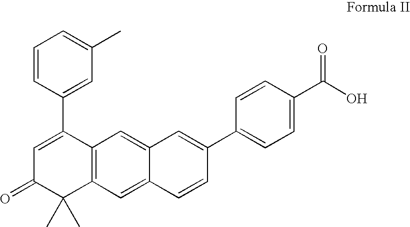

This compound can be synthesized routinely by those of skill in the art in accordance with well known methods. An exemplary method of synthesis for this compound is set forth in Example 2. Further, as will be understood by those of skill in the art upon reading this disclosure, other compounds exhibiting RAR pan antagonistic activity can also be used. Additional examples of RAR pan antagonists are disclosed in WO 97/48672. Other compounds with this activity can also be identified routinely by those of skill in the art in accordance with methods such as described in WO 97/48672.

In addition to their free acid forms, pharmaceutically acceptable salts of the compounds of Formulas I, Ia, Ib and II are useful in the methods and compositions of the present invention. Such pharmaceutically acceptable salts include, but are not limited to, salts formed with alkali metals such as sodium, potassium and lithium; alkaline earth metals such as calcium and magnesium; organic bases such as dicyclohexylamine, tributylamine, pyridine and the like; and amino acids such as arginine, lysine and the like.

By “RAR pan agonist” it is meant a compound which activates the α, β and γ isotypes of the retinoic acid receptors. Examples of RAR pan agonists include, but are not limited to, ATRA, 13cis retinoic acid, and ALRT1550. While RAR pan agonists have been reported to inhibit tumor progression, these treatments have been limited by unwanted toxicities including, but not limited to, mucocutaneous toxic effects, headaches, teratogenesis, musculoskeletal toxicity, dylipidemias, skin irritation and hepatotoxicity. The mucocutaneous toxic effects of RAR pan agonists, which are considered as the major toxicity induced by these compounds and limits their use as therapeutic agents, are associated with RARγ activity. Further, the natural retinoid ATRA is converted in situ into 9cis-RA which is a pan RAR and RXR agonist. Activation of RXR following ATRA administration has also been associated with undesirable side effects.

By “selected cytotoxic agent”, it is meant a taxane or other agent which acts by similar mechanisms to taxanes. Accordingly, this term is meant to be inclusive of other tubulin polymerizing agents. Also, for synergistic compositions comprising a RARα/β selective agonist, this term is meant to be inclusive of other agents which increase Bcl-2 phosphorylation. In a preferred embodiment, the selected cytotoxic agent is paclitaxel or docetaxel.

As demonstrated herein, RARα/β selective agonists and RAR pan antagonists act synergistically with selected cytotoxic agents to lower the effective dose of the selected cytotoxic agent required to induce cytotoxicity in tumor cells. In one embodiment of the present invention, the synergistic combination comprises a RARα/β selective agonist in combination with a second agent which increases phosphorylation of Bcl-2, such as a tubulin polymerizing agents, preferably a taxane. In another embodiment of the present invention, the synergistic combination comprises a RAR pan antagonist and a tubulin polymerizing agent, preferably a taxane.

It is believed that these synergistic combinations will be effective in treating cancer and reducing toxic side effects associated with the selected cytotoxic agents of the combination. Further, use of RARα/β selective agonists or RAR pan antagonists in the combinations of the present invention eliminates or decreases toxic side effects associated with activation of RARγ and RXR following administration of RAR pan agonists and ATRA. Accordingly, the present invention also relates to methods of treating cancer by administering to a patient a RARα/β selective agonist or RAR pan antagonist in combination with a selected cytotoxic agent. In these methods, it is preferred that the RARα/β selective agonist or RAR pan antagonist be administered in combination with a tubulin polymerizing agent, most preferably a taxane. Alternatively, for RARα/β selective agonists, the selected cytotoxic agent may also comprise an agent which increases phosphorylation of Bcl-2.

By “cancer” it is meant to include tumors, neoplasias, and any other malignant tissue or cells.

The present invention also relates to pharmaceutical compositions comprising a selected cytotoxic agent and a RARα/β selective agonist or RAR pan antagonist. In a preferred embodiment, the selected cytotoxic agent comprises a tubulin polymerizing agent, most preferably a taxane. Alternatively, for compositions comprising a RARα/β selective agonist, the selected cytotoxic agent may also comprise an agent which increases phosphorylation of Bcl-2.

The compositions of the present invention may comprise both components in a single pharmaceutically acceptable formulation. Alternatively, the components may be formulated separately and administered in combination with one another. Various pharmaceutically acceptable formulations well known to those of skill in the art can be used in the present invention. Selection of an appropriate formulation for use in the present invention can be performed routinely by those skilled in the art based upon the mode of administration and the solubility characteristics of the components of the composition.

For purposes of the present invention, by “combination” it is meant the RARα/β selective agonist or RAR pan antagonist is administered to the patient prior to, simultaneously with, or following administration of the selected cytotoxic agent. In a preferred embodiment, the RARα/β selective agonist or the RAR pan antagonist is administered to the patient prior to or simultaneously with the selected cytotoxic agent. As used herein, the term “simultaneous” or “simultaneously” means that the RARα/β selective agonist or the RAR pan antagonist and the selected cytotoxic agent are administered within 24 hours, preferably 12 hours, more preferably 6 hours, and most preferably 3 hours or less, of each other.

The ability of RARα/β selective agonists and RAR pan antagonists to additively or synergistically effect cell growth when administered in combination with existing chemotherapeutic agents was first assessed in three different human tumor cell lines. Inhibition of cell growth was compared in cells co-treated with paclitaxel and either a RAR pan agonist (see Formula III), the RARα/β selective agonist of Formula Ia, an RARα agonist (see Formula IV), an RAR β agonist (see Formula V), an RAR γ agonist (see Formula VI), the RAR pan antagonist of Formula II and a RXR pan agonist (see Formula VII). The structure of Formula III through VII are depicted below:

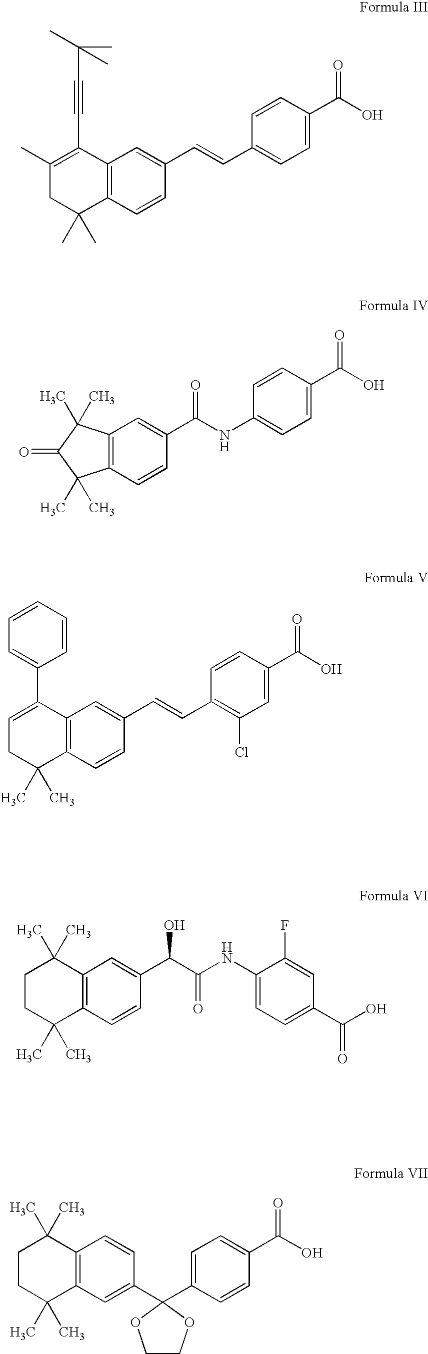

As shown in Table 1, the RARα/β selective agonist of Formula Ia dramatically lowered the effective dose of paclitaxel required to induce cytotoxicity. In the presence of Formula Ia, the IC₅₀ of paclitaxel was decreased by 8-, 15- and 32-fold in MCF7, OVCAR3 and SQCC-Y1 cells, respectively. These values were very similar to those obtained for the combination of paclitaxel with the RAR pan agonist of Formula III, thus supporting a major role of RARα/β isotypes in the tumor-suppressive activity of the retinoids. The RAR pan antagonist of Formula II also lowered the effective dose of paclitaxel required to induce cytotoxicity. In contrast, the pure RARα agonist (Formula IV) was associated with only a 2- to 4-fold decrease in the IC₅₀ of paclitaxel (Table 1), which is in agreement with its low affinity for RARα compared to Formula Ia. Neither the pure agonist for RARβ (Formula V), the RAR-γ-selective agonist (Formula VI), nor the pure RXR agonist (Formula VII), effected paclitaxel cytotoxicity by more than 2- to 3-fold, thus indicating that the synergistic effects of the RARα/β ligands with paclitaxel is mainly mediated by RARα (Table 1). One exception however is represented by the γ-selective agonist in OVCAR3 cells where it lowered the IC₅₀ of paclitaxel by 8-fold.

TABLE 1
Selectivity required for retinoid/paclitaxel
synergistic inhibition of cell growth
Growth Inhibition ([³H]-Thymidine Incorporation)

paclitaxel

| | Selectivity | IC50 | Efficacy |
|---|---|---|---|
| MCF7 | none | 0.070 | 100 |
| | RAR pan ago. | 0.008 | 100 |
| | (Formula III) | | |
| | RARα/β ago. | 0.009 | 98 |
| | (Formula Ia) | | |
| | RARα ago. | 0.017 | 100 |
| | (Formula IV) | | |
| | RARβ ago. | 0.064 | 100 |
| | (Formula V) | | |
| | RARγ ago. | 0.044 | 100 |
| | (Formula VI) | | |
| | RAR pan atg. | 0.007 | 100 |
| | (Formula II) | | |
| | RXR pan ago. | 0.068 | 100 |
| | (Formula VII) | | |
| SQCC-Y1 | none | 0.160 | 100 |
| | RAR pan ago. | 0.008 | 100 |
| | (Formula III) | | |
| | RARα/β ago. | 0.005 | 100 |
| | (Formula Ia) | | |
| | RARα ago. | 0.05 | 100 |
| | (Formula IV) | | |
| | RARβ ago. | 0.113 | 100 |
| | (Formula V) | | |
| | RARγ ago. | 0.088 | 100 |
| | (Formula VI) | | |
| | RAR pan atg. | 0.037 | 100 |
| | (Formula II) | | |
| | RXR pan ago. | 0.111 | 100 |
| | (Formula VII) | | |
| OVCAR3 | none | 0.6 | 99 |
| | RAR pan ago. | 0.080 | 99 |
| | (Formula III) | | |
| | RARα/β ago. | 0.040 | 100 |
| | (Formula Ia) | | |
| | RARα ago. | 0.251 | 100 |
| | Formula IV) | | |
| | RARβ ago. | 0.2 | 100 |
| | (Formula V) | | |
| | RARγ ago. | 0.079 | 100 |
| | (Formula VI) | | |
| | RAR pan atg. | 0.100 | 100 |
| | (Formula II) | | |
| | RXR pan ago. | 0.22 | 100 |
| | (Formula VII) | | |

In this table, efficacy is defined as percent (%) inhibition compared with DMSO. All IC₅₀'s are expressed in nanomolar values. The results are the mean of 2 to 5 experiments. The standard deviation associated with the IC₅₀'s ranges between 10 and 20%.

Additional experiments were performed to compare the ability of MCF7 cells to form colonies in the presence of a selective RARα/β agonist, a RAR pan antagonist or ATRA. The colonies grown in the presence of the selective RARα/β agonist of Formula Ia clearly revealed the differentiating effect of the retinoid. The vehicle DMSO-treated cells formed very compact colonies, the cells showed a very restricted cytoplasm and most of the colonies appeared very well delineated. In contrast, the treatment of MCF7 cells with the RARα/β agonist of Formula Ia resulted in a dramatic change in the morphology of both the colonies and the cells. The colonies appeared dispersed, the cells gained in volume, exhibiting a cytoplasm, and tended to organize into an epithelium-like structure. Treatment of the MCF7 cells with a RAR pan antagonist, Formula II, also strongly impaired colony formation. The colonies remained small with two apparent populations of cells, one apoptotic and one in a process of differentiation, showing plasma extension. Further, pre-treatment of these cells for three days with the RAR pan antagonist significantly enhanced the sensitivity of the cells to paclitaxel. ATRA also impaired colony formation. However, in contrast to RARα/β selective agonists, ATRA-treated colonies exhibited a high level of apoptosis which is believed to reflect the activity of RXR consecutive to the conversion in situ of ATRA to 9cis-RA.

The effects of both paclitaxel and RARα/β selective agonists or RAR pan antagonists on the signal transduction pathways common to these agents were analyzed. In these experiments, the effects of paclitaxel/retinoid co-treatment on the expression levels and phosphorylation status of the anti-apoptotic protein, Bcl-2, a protein implicated in both retinoid and paclitaxel signaling were first examined. MCF7 cells treated with paclitaxel displayed a dose-dependent increase in the phosphorylation of Bcl-2. The addition to the cells of a selective α/β-agonist resulted in a down-regulation of the Bcl-2 protein levels, while the RAR pan antagonist alone had no effect on Bcl-2 levels or phosphorylation. The combination of paclitaxel with the selective RAR agonist resulted in a down-regulation of Bcl-2 levels and an increase in its phosphorylation status. In contrast, the combination of paclitaxel with an RAR pan antagonist was only associated with an increase in the phosphorylation of Bcl-2 without modulation of its levels. These results indicate that the synergistic effect of paclitaxel and RARα/β selective agonists could, in part, be due to additive effects at the levels of down-regulation and phosphorylation of Bcl-2 that results in the release of pro-apoptotic Bax activity, mitochondria membrane permeabilization and the induction of apoptosis. Thus, it is believed that other agents that increase Bcl-2 phosphorylation in similar fashion to paclitaxel will exhibit similar synergistic activity with RARα/β selective agonists.

In contrast, the RAR pan antagonist did not effect Bcl-2 protein levels or paclitaxel-induced phosphorylation of Bcl-2, thus indicating that the synergy between tubulin polymerizing agents such as paclitaxel and retinoid antagonists may use a different pathway.

The combination effect of paclitaxel and RARα/β agonists or RAR pan antagonists on tubulin polymerization was also analyzed. Pools of soluble, non-polymerized tubulin were separated from the insoluble polymerized tubulin pool and analyzed by Western blotting using anti-αtubulin antibodies. A dose-dependent polymerization of tubulin induced by paclitaxel was observed. However, the selective RAR agonists or antagonists had no effect. Further, the combination of paclitaxel with any of the retinoids did not enhance the effect of paclitaxel alone. Thus, the retinoids do not appear to synergize with paclitaxel's effects on the cytoskeleton to induce cytotoxicity.

Several recent reports have described Taxol-induced stimulation of Jun N-terminal Kinase (JNK) activity, leading to an increase in c-Jun phosphorylation and associated increase in activity of the AP1 transcription complex (Lee et al. J. Biol. Chem. 1998 273:28253-28260; Wang et al. J. Biol. Chem. 1998 273:4928-2936). Retinoids have also been shown to regulate and inhibit AP1 transcriptional activity (Chen et al. EMBO J. 1995 14:1187-1197). In this context, the combined effect of paclitaxel and retinoids on JNK kinase activity and AP1 transcriptional activity in MCF7 cells was analyzed. The phosphorylation of c-Jun by immuno-precipitated JNK revealed that the treatment of MCF7 cells by paclitaxel resulted in an average of 2.5 fold increase in JNK activity, while the retinoids alone had no effect on JNK activity. In contrast, the combination of paclitaxel with a α/β selective agonist, slightly strengthened paclitaxel-induced JNK activity yielding a maximum of 4-fold increase in enzymatic activity. These effects on JNK were further analyzed at the level of the transcription complex activity by using a stable MCF7 cell line stably expressing the AP1 reporter gene, (AP1)5x-tk-Luc. The potent activity of this reporter construct induced by phorbol ester (PMA) in MCF7 cells was inhibited in a dose-dependent manner by the selective RARα/β agonist (Formula Ia), reaching a maximum of 30 to 45% at 50 nM with the retinoid. In contrast, paclitaxel further enhanced by 20% PMA-induced AP1 transcriptional activity when added as a single agent. The combination of paclitaxel with the retinoid prevented the paclitaxel effect on PMA-induced collagenase promoter activity resulting in 30 to 50% inhibition of the AP1 transcription factors activity in response to the combination of paclitaxel and PMA. Thus, the combination of paclitaxel with RAR α/β selective agonists enhances the paclitaxel-effect on the Jun-N-terminal kinase activity which, however, becomes uncoupled from the subsequent AP1 transcriptional activation due to the negative cross-regulation of AP1 induced by the retinoids. These observations, together with the effects on Bcl-2 may underlie the potent synergistic cytotoxic effect of these agents. The combination of paclitaxel with the RAR pan antagonist of Formula II did not modify paclitaxel-induced JNK activity. However, this combination did enhance the effects of paclitaxel/PMA or AP1 activity.

The following nonlimiting examples are provided to further illustrate the present invention.

### EXAMPLES

### Example 1

### Materials

The cell lines used in these experiments including T47D, HT-3, UMSCC-25, MCF-7, OVCAR3, HCT-1 16, and N-87 were obtained from ATCC and maintained in the media and serum concentration recommended by ATCC. H3396 cell line was obtained in accordance with the procedure set forth by Garrigues et al. (Am. J. Pathol. 1993 142:607-622), and maintained in RPMI supplemented with 10% FBS (Gibco-BRL). MDA-PCA.2B and SQCC-YI were obtained from the Anderson Cancer Center (Houston, Tex.). A2780S and its p27 transgenic variant A278Op27, HCT-116TYPK (Taxol-resistant) and HCTVM46, SAN-1, M109 and its Taxol-resistant variant M109TX, and PAT-7 (Taxol resistant) cell lines engineered for drug resistance were also used. All the cytotoxic agents and phorbol 12-myristate 13-acetate (PNIA) were purchased from Sigma-Aldrich. The mouse anti-αBcl-2, anti-α-tubulin, and anti-human JNK antibodies used in these experiments were purchased from Biosource International, Sigma-Aldrich and BD-Pharmingen, respectively. The c-jun (1-169)-GST fusion protein was obtained from Upstate Biotechnology.

### Example 2

### Synthetic RAR Ligands

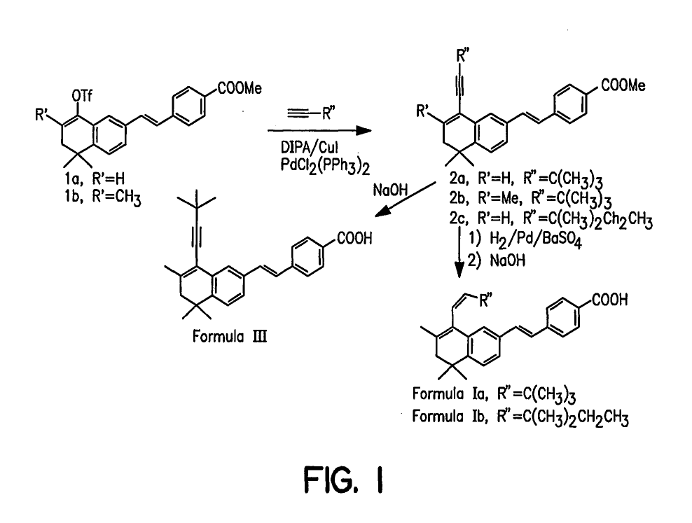

The routes of synthesis and structures of the RAR α/β selective agonists and the RAR used in these experiments are outlined in FIG. 1. Synthesis of the dihydronaphthalenyl compounds Formula Ia, Formula Ib and Formula III involves a Sonogashira coupling of the desired enol triflates 1a or 1b with various acetylenes. Compounds 2a and 2b were generated in the presence of 3,3-dimethyl-butyne, diisopropylamine, copper(I) iodide, and bis(triphenylphosphine)palladium(II) chloride. Similarly, 3,3-dimethyl-pent-l-yne (Van Boom et al. Rec. Trav. Chim. 1965 84:31) was used to prepare compound 2c. The triple bond in 2a and 2c was hydrogenated using 10% palladium on barium sulfate and subsequently saponified at the methyl ester moiety to produce the desired cis-ethylene derivatives Formula Ia and Formula Ib. Similarly, the RAR pan agonist of Formula III was synthesized through the saponification of the acetylene 2b.

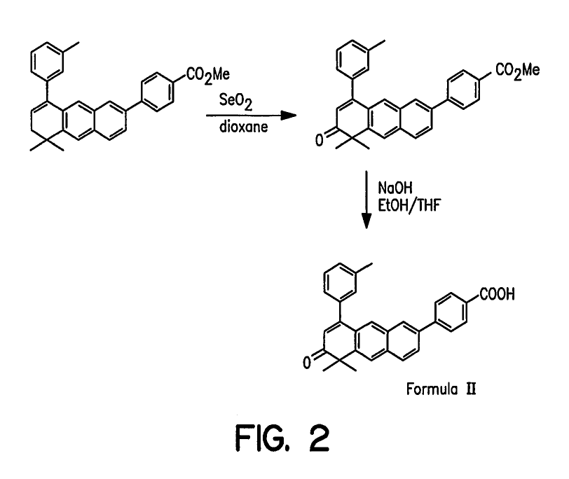

The RAR pan antagonist of Formula II, 4-(5,6-dihydro-5,5-dimethyl-6-oxo-8-(3-methyl-phenyl)-anthracen-2-yl)-benzoic acid was prepared in accordance with the synthesis outlined in FIG. 2 and described below.

### Methyl 4-(5,6-dihydro-5,5-dimethyl-6-oxo-8-(3-methyl-phenyl)-anthracen-2-yl)-benzoate

A stirred solution of methyl 4-(5,6-dihydro-5,5-dimethyl-8-(3-methyl-phenyl)-anthracen-2-yl)-benzoate (0.181 g, 0.418 mmol) in dioxane (6 mL) was treated with selenium dioxide (0.186 g, 1.672 mmol) and the mixture was refluxed for 5 hours. The reaction mixture was then diluted with ethyl acetate and washed with brine, dried over magnesium sulfate, filtered and concentrated. The residue was purified using silica gel chromatography to give the title material (0.155 g, 83%).

¹H NMR 400 MHZ C₆D₆ δ ppm: 1.69(6H,s,2x-CH₃),2,14(3H,s,—CH₃)6.38 (1H,s,H-7′),7.03-7.19(3H,m,aromatic H),7.33(2H,J=8.4 Hz, H-3 and H-5),7.48-7.49(2H,m,aromatic H),7.59(1H,d,J=9.1 Hz,aromatic H), 7.78(1H,s,aromatic H),7.89 1H,s,aromatic H),8.24(2H,d, J=8.4 Hz, H-2 and H-6).

### 4-(5,6-Dihydro-5,5-dimethyl-5-oxo-8-(3-methyl-phenyl)-anthracen-2-yl)-benzoic acid

A stirred solution of methyl 4-(5,6-dihydro-5,5-dimethyl-6-oxo-8-(3-methyl-phenyl)-anthracen-2-yl)-benzoate (0.015 g, 0.034 mmol) in ethanol (2 mL) and tetrahydrofuran (2 mL) was treated with an aqueous sodium hydroxide solution (5N, 0.5 mL) and the mixture was stirred overnight at room temperature. The solvents were then evaporated and the residue was diluted with ethyl acetate (4 mL) and acidified with aqueous hydrochloric acid 1N. The organic layer was washed with brine and dried over anhydrous magnesium sulfate, filtered and concentrated.

¹H NMR 400 MHZ DMSO-d₆ δ ppm: 1.59 (6H,s,2x -CH3),2.43(3H,s, —CH3),6.12(1H,s,H-7′),7.35-7.39, 7.46-7.50(3H and 1H, 3 sets of m,aromatic H),7.91(1H,s,aromatic H), 7.96-8.04 (5H,m,aromatic H), 8.09(1H,d,J=8.6 Hz,aromatic H), 8.25 and 8.34 (1H and 1H,2s, aromatic H).

### Example 3

### Mitogenic Assay

Cellular proliferation was quantified using [³H] thymidine incorporation. Cells were plated in 96 well plates in media containing 10% fetal bovine serum (FBS) and allowed to adhere overnight. Cell medium was removed the following day and the indicated compounds were added to fresh medium supplemented with 10% of charcoal stripped FBS (HyClone). Compound-containing medium was renewed after 3 days exposure. After 6 day treatment, 4 μCi/ml of [³H]thymidine (NEN Life Science Products) were added to each well for 2 to 4 hours. The cells were then trypsinized and harvested on GF/B glass fiber filters. [³H]thymidine incorporated into the DNA was measured in a scintillation Topcount counter (Packard). The results were expressed as percent inhibition compared to the DMSO vehicle. IC₅₀ was defined as the concentration of compound inducing 50% inhibition and the efficacy corresponded to the maximal inhibition obtained in response to the compound and compared to DMSO. The results are the mean of 2 to 5 independent experiments.

### Example 4

### Anchorage-dependent Colony Formation Assay

To determine effects of RAR agonists on anchorage-dependent colony size, number and morphology, MCF7 cells were plated in 6 well plates (500 cells/well) in DMEM supplemented with 10% FBS and grown for 5 days. The medium was then removed and the compounds were added to the medium supplemented with 5% of charcoal stripped FBS (HyClone). The compound containing medium was renewed after 3 days and cultures were allowed to grow an additional 3 days. After 6 days exposure to compounds, colonies were stained with crystal violet. Colonies were counted to quantify colony area and number and photographed under light microscopy to assess effects on colony size and morphology. The results presented are representative of two independent experiments. Immunoblotting MCF-7 cellular lysates were prepared for Western blotting in accordance with procedures described by Sambrook et al. (MOLECULAR CLONING: A LABORATORY MANUAL, 2nd Ed.; Cold Spring Harbor Laboratory Press, Cold Spring Harbor, N. Y. (1989)). Cell lysates were prepared by adding 200 μl of lysis buffer (Tris-HCl, pH 6.8, 20 mM, EDTA 1 mM, NP-40 0.5%) containing a protease inhibitor cocktail (Roche) to each well of 6 well plates. The lysates were then cleared by centrifugation at 10,000 rpm, 4EC for 5 minutes and supernatants were adjusted with Laemmli sample buffer and processed by 10 or 15% SDS-polyacrylamide gel electrophoresis (PAGE), followed by transfer to nitrocellulose membranes (Biorad). Membranes were processed by standard methods and incubated with primary antibodies for 1 hour at room temperature or overnight at 4EC. Bound antibody was visualized using a goat anti-IgG secondary antibody conjugated to HRP and a chemiluminescent detection system (ECL, Amersham Pharmacia Biotech).

### Example 5

### Tubulin Polymerization Assay

The effects of retinoid and paclitaxel exposure on free and polymerized tubulin levels were determined in accordance with procedures described by Giannakakou et al. (J. Biol. Chem. 1997 272:17118-17125). MCF7 cells were plated in 6 well plates in DMEM supplemented with 10% FBS and allowed to adhere overnight. Cell medium was removed the following day and the indicated compounds were added to fresh medium supplemented with 5% of charcoal stripped FBS (HyClone). The cells were grown for 3 days in the presence of the retinoids before paclitaxel was added for 1 hour. Soluble and polymerized tubulin were extracted and respective levels were assessed by Western blotting as described above using anti-(tubulin antibodies (n=2).

### Example 6

### AP1 Reporter Assay

AP1 transcriptional activity was assessed in MCF7 cells stably transfected with (AP1)5x-tk-Luc reporter gene (IGBMC, France). (AP1)5x corresponds to the collagenase gene AP1 response element repeated 5 times in tandem, tk is the minimal thymidine kinase promoter, and Luc the coding sequence of the luciferase gene. MCF7 cells were plated in 12-well plates at a density of 400,000 cells per well in DMEM supplemented with 10% FBS and allowed to adhere overnight. The next day, the medium was removed, the cells were washed with PBS and supplemented with DMEM medium containing 0.5% of charcoal stripped FBS (HyClone). Twenty-four hours later, the cells were treated for 6 hours with PMA (10 nM) in the absence or the presence of ATRA, Formula Ia or a RAR pan agonist of Formula III (concentrations ranging from 0.1, 1 and 50 nM) combined with or without paclitaxel (1 μM). The luciferase activity was measured by using Luc-lite system from Packard according to manufacturer's recommendations. Results are expressed as percent activity compared to PMA (10 nM) which was assigned as 100% activity. The data presented are the mean ± sem (standard error of the mean)of 3 independent experiments.

### Example 7

### Jun N-terminal Kinase Assay

Activity of JNK was measured in accordance with procedures described by Coso et al. (Cell 1995 81:1137-1146) with minor modifications. MCF-7 cells were plated in 6-well plates in DMEM supplemented with 10% FBS and allowed to adhere overnight. The next day, the cells were switched to DMEM supplemented with 0.5% charcoal-stripped FBS (HyClone) and 100 nM of indicated retinoids or 0.1% DMSO was added to the cells for 24 hours before an additional 2 to 3 hours incubation in the presence of 1 μM of paclitaxel. Cell lysates was prepared from each well as described and cleared by centrifugation. JNK was immunoprecipitated from each lysate by incubating overnight at 4° C. with 1 μg of anti-hJNK (Pharmingen) and 25 μl of protein G agarose beads (Gibco-BRL) previously washed with the lysis buffer. Immune complexes were pelleted by centrifugation 2 minutes at 10,000 rpm 4° C. and washed 3 times with PBS containing 1% NP40 and 2 mM of Na₃VO₄, once with 100 mM Tris-HCl pH7.5, 0.5 mM LiCl and once with Kinase Reaction Buffer (KRB: 12.5 mM MOPS pH7.5, 12.5 mM β-glycerolphosphate, 7.5 mM MgCl₂, 0.5 mM EGTA, 0.5 mM NaF, 0.5 mM Na₃VO₄). Kinase assays were performed at 30° C. for 30 minutes by resuspending the beads in 30 μl of KRB containing 5 μCi [gamma- ³³P]ATP (NEN Life Science Products), 10 μM cold ATP, 3.3 mM DTT and 2 μg of GST-c-Jun(1-169) fusion protein. The kinase reaction was terminated by adding 10 μl of Laemmli sample buffer. resolved by 10% SDS-PAGE and phosphorylated GST-c-Jun visualized by autoradiography and quantified by densitometry. The results represent the mean ± sem of 3 independent experiments.

What is claimed is:

1. A method of treating cancer in a patient which comprises administering to a patient in need thereof:
a) a selected cytotoxic agent; and
b) a RARα/β selective agonist or a RAR pan antagonist.

2. The method of claim 1 wherein the selected cytotoxic agent is a tubulin polymerizing agent.

3. The method of claim 1 wherein the selected cytotoxic agent is a taxane.

4. The method of claim 1 wherein the selected cytotoxic agent is paclitaxel.

5. The method of claim 1 wherein the RARα/β selective agonist or the RAR pan antagonist is administered to the patient prior to or simultaneously with the selected cytotoxic agent.

6. The method of claim 1 which comprises administering:
a) a selected cytotoxic agent which increases phosphorylation of Bcl-2; and
b) a RARα/β selective agonist.

7. The method of claim 1 wherein the RARα/β selective agonist is selected from the group consisting of Formula Ia and Formula Ib and pharmaceutically acceptable salts thereof.

8. The method of claim 1 wherein the RAR pan antagonist is selected from the group conisting of Formula II and pharmaceutically acceptable salts thereof.

9. A method for lowering the effective dose of a selected cytotoxic agent required to induce cytotoxicity in tumor cells comprising administering to the cells:
a) the selected cytotoxic agent; and
b) a RARα/β selective agonist or a RAR pan antagonist.

10. The method of claim 9 wherein the selected cytotoxic agent is a tubulin polymerizing agent.

11. The method of claim 9 wherein the selected cytotoxic agent is a taxane.

12. The method of claim 9 wherein the selected cytotoxic agent is paclitaxel.

13. The method of claim 9 wherein the RARα/β selective agonist is selected from the group consisting of Formula Ia and Formula Ib and pharmaceutically acceptable salts thereof.

14. The method of claim 9 wherein the RAR pan antagonist is selected from the group consisting of Formula II and pharmaceutically acceptable salts thereof.

15. The method of claim 9 which comprises administering:
a) a selected cytotoxic agent which increases phosphorylation of Bcl-2; and
b) a RARα/β selective agonist.

16. A pharmaceutical composition comprising:
a) a selected cytotoxic agent; and
b) a RARα/β selective agonist or a RAR pan antagonist.

17. The pharmaceutical composition of claim 16 wherein the selected cytotoxic agent is a tubulin polymerizing agent.

18. The pharmaceutical composition of claim 16 wherein the selected cytotoxic agent is a taxane.

19. The pharmaceutical composition of claim 16 which comprises:
a) a selected cytotoxic agent which increases phosphorylation of Bcl-2; and
b) a RARα/β selective agonist.

20. A compound of Formula II or a pharmaceutically acceptable salt thereof.
